(19) United States
(12) Patent Application Publication
(10) Pub. No.: US 2009/0287117 A1
(43) Pub. Date: Nov. 19, 2009

(54) PUNCTURING DEVICE

(76) Inventors: Herbert Harttig, Neustadt (DE); Hans List, Hesseneck-Kailbach (DE)

Correspondence Address: BOSE MCKINNEY & EVANS LLP, 111 Monument Circle, Suite 2700, INDIANAPOLIS, IN, 46204, US

(21) Appl. No.: 12/480,265

(22) Filed: Jun. 8, 2009

Related U.S. Application Data
(63) Continuation of application No. PCT/EP2007/010107, filed on Nov. 22, 2007.

(30) Foreign Application Priority Data
Dec. 8, 2006 (EP) EP 06025376.2

(51) Int. Cl. A61B 5/00; A61B 17/34

(52) U.S. Cl. 600/584; 606/185

(57) ABSTRACT

The invention relates to a piercing device for creating a puncture wound for collecting a sample of body fluid, comprising a rubber elastic pressure part to be pressed onto a body part from which a sample of body fluid is to be removed, and a piercing drive mechanism by means of which a piercing element inserted into the piercing device can be driven in a piercing movement. According to the invention, an electrical deformation sensor is provided for detecting an elastic deformation of the pressure part by means of an electrical and/or magnetic measurement.

### RELATED APPLICATIONS

This application is a continuation application of International Application PCT/EP2007/010107, filed Nov. 22, 2007, which claims priority to EP 06025376.2, filed Dec. 8, 2006, which are hereby incorporated by reference in their entirety.

### BACKGROUND

The invention relates to a puncturing device for generating a puncturing wound for obtaining a sample of body fluid, comprising a rubber-elastic press-against part to be pressed against a body part from which a sample of body fluid is to be taken, and a puncturing element drive for driving a puncturing element that is inserted in the puncturing device in order to perform a puncturing motion. Puncturing devices of this type are known from WO 01/89383 and are used, in particular, by diabetics in order to determine blood sugar concentration.

Puncturing devices having a rubber-elastic press-against part are advantageous in that they can conform to body parts of various shapes and, in the process, exert pressure on body tissue around the puncturing wound and transverse to the puncturing direction. By this means, the escape of body fluid from a newly made puncturing wound can be promoted and obtaining a sample can thus be made easier for a user.

In the puncturing device known from WO 01/89383, the press-against force applied by the user to the press-against part is monitored by means of a combination of a spring and a limit switch. This ensures that a puncture and an ensuing measurement are performed only if the user applies a minimum press-against force that is sufficient for obtaining a sample on the press-against part.

A disadvantage of the known puncturing device having a mechanical press-against sensor as described above is that, although it allows determining whether the press-against force generated by a user exceeds a minimum pressure required for obtaining a sample, it does not allow a press-against force that is unfavorably large for obtaining a sample to be recognized at all or only by extensive use of equipment. Excessive press-against forces can obstruct a puncturing channel that is made and may also force body fluid away from the tissue surrounding the puncturing wound such that it is more difficult to obtain a sample.

Another disadvantage of mechanical press-against sensors that are based on a combination of a spring and a switch is that contamination of the switch contacts might adversely affect reliability and lead to failure. In particular, the replacement of the rubber-elastic press-against elements, which is to be performed regularly for hygienic reasons, is associated with an increased risk of contaminating switch contacts. This requires the user to exercise more care during the replacement of the rubber-elastic press-against parts, which is strenuous and considered stressful, especially by users whose motor skills are limited due to age or disease.

### SUMMARY OF THE INVENTION

The present invention provides a means of reducing the risk of an unsuccessful puncture due to the body part being incorrectly pressed against the press-against part. To accomplish this, an electrical deformation sensor is provided for detecting an elastic deformation of the press-against part by an electrical and/or magnetic measurement.

The inventors recognized that the nature and extent of the deformation of the press-against part of a puncturing device having a rubber-elastic press-against part is significantly more important for the success of obtaining a sample than the numerical value of the pressure that is applied against the press-against part. Only sufficient deformation of the press-against part allows the part to conform to a body part that is pressed against it and to thus surround a bulge of the body part in the region in which the puncturing wound is to be generated such that an increased body fluid pressure is generated in this place that supports the escape of body fluid from the puncturing wound. Accordingly, use of a deformation sensor allows for a more reliable determination of whether favorable conditions for obtaining a sample are present.

An electrical deformation sensor that is used according to certain embodiments has the additional advantage that it is significantly less susceptible to contamination than mechanical pressure sensors having limit switches. Since a puncturing device having an electrical deformation sensor does not need open switch contacts, there is no risk of such switch contacts getting contaminated, especially by blood. Moreover, having an electronic deformation sensor allows an electrical sensor signal to be generated that contains not only information regarding whether the deformation of the press-against part exceeds a minimum deformation that is required for taking a sample, but also additional information regarding whether or not the deformation of the press-against part exceeds a damaging upper limit. A puncture made under excessive pressure that presses body fluid away from the region of a body part that is intended for obtaining a sample or obstructs a puncturing channel made can thus be prevented in a puncturing device according to the invention.

### BRIEF DESCRIPTION OF THE DRAWINGS

The above-mentioned aspects of the present invention and the manner of obtaining them will become more apparent and the invention itself will be better understood by reference to the following description of the embodiments of the invention, taken in conjunction with the accompanying drawings, wherein:

FIG. 1 shows a schematic exemplary embodiment of a puncturing device according to the invention with a body part being pressed against the press-against part;

FIG. 2 shows the press-against part of the exemplary embodiment shown in FIG. 1 at lower press-against pressure; and

FIG. 3 shows a perspective view of the press-against part of the exemplary embodiment shown in FIG. 1 in the absence of deformation.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

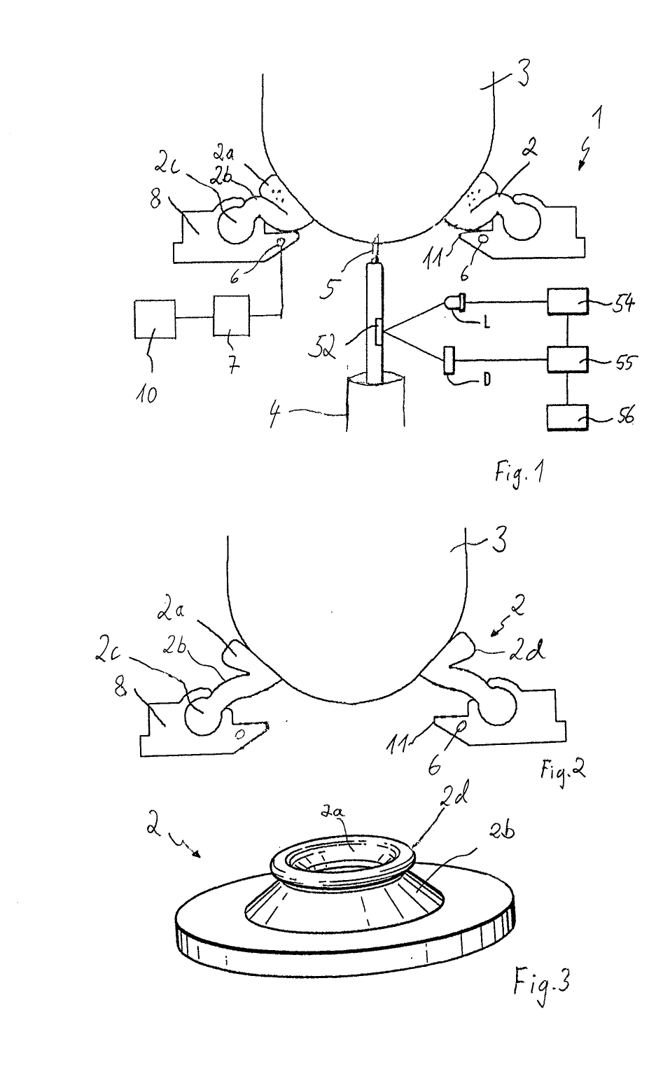

FIG. 1 shows a schematic view of an exemplary embodiment of a puncturing device 1 for generating a puncturing wound for obtaining a sample of body fluid, comprising a rubber-elastic press-against part 2 to be pressed against a body part 3 from which the sample of body fluid is to be obtained, and a puncturing element drive 4 for driving a puncturing element 5 to perform a puncturing motion. The rubber-elastic press-against part is provided as a press-against cone that increases the pressure of the body fluid in a body part 3 that is pressed against it and thus favors the escape or leakage of body fluid from a puncturing wound that has been produced with a puncturing element 5 that is inserted into the puncturing device 1.

In order for the rubber-elastic press-against part 2 to conform well to a body part 3 that is pressed against it, it has a hardness of less than 90 Shore-A, in particular less than 50 Shore-A. A Shore hardness in the range from 20 to 40 Shore-A is particularly favorable. Aside from elastic plastic materials, such as, for example, polyurethane, silicones and rubber are also suitable materials for the press-against part 2.

Shown in FIGS. 2 and 3 in a less deformed state of the press-against part and without deformation, respectively, the press-against part 2 has two conically extending regions 2a, 2b, whereby the upper region 2a forms a press-against region that narrows in the direction of pressing. When a body part 3 is pressed against the press-against part 2, body tissue bulges into the press-against region 2a and becomes surrounded by the press-against part 2 conforming to the body tissue such that an elastic pressure is applied transverse to the direction of pressing and effects an increase of the pressure of the body fluid in the region in which the puncturing wound is to be generated.

A lower region 2b is adjacent to the press-against region 2a and widens in the direction of pressing. The press-against part 2 has a clamping rim 2c for attachment in a bracket 8 of the puncturing device 1 and a rim 2d on the upper edge that is seen particularly well in FIG. 3. More details of a rubber-elastic press-against part of this type are disclosed in WO 01/89383 A2 which is incorporated in the present application by way of reference.

An electrical deformation sensor 6 for detecting an elastic deformation of the press-against part 2 is an important particularity of the exemplary embodiment shown. In the exemplary embodiment shown, a total of four such deformation sensors 6 are provided and are distributed over the circumference, each of which measures the elastic deformation of a partial region of the press-against part 2. If multiple deformation sensors 6 are utilized, as is the case in the exemplary embodiment shown, it can be determined if the body part 3 is in an orientation with respect to the puncturing device 1 pressed against it that is unfavorable for obtaining a sample such that the risk of unsuccessful punctures that do not lead to a usable sample of body fluid can be markedly reduced.

The deformation sensor 6 uses an electrical and/or magnetic measurement to generate a sensor signal that depends in a continuous way on the deformation of the press-against part 2. By this means, it can be determined whether the deformation of the press-against part 2 caused by pressing against it the body part 3 from which a sample of body fluid is to be taken is in a favorable range. This is significant since too little deformation insufficiently increases the pressure of the body fluid in the body part 3 in the region of the puncturing wound to be made for obtaining a sample with as little pain as possible. On the other hand, a deformation that is too extensive might force body fluid away from the region of the puncturing wound, such that obtaining a sample may be more difficult also if the deformation is too extensive.

In the exemplary embodiment shown, the deformation sensor 6 detects a magnetic field change that is connected to the deformation of the press-against part 2. The press-against part 2 contains a magnetic additive, for example, ferrite particles or any other ferro- or ferrimagnetic additive. Magnetic particles can be admixed to a plastic material from which the press-against part is made without difficulty and magnetized permanently such that a deformation of the press-against part 2 is associated with a magnetic field change that can be detected by the deformation sensor 6. The deformation sensor 6 can, for example, be a Hall sensor. Inductive sensors are also suitable. An inductive sensor contains a resonant circuit with a coil. The inductance of this coil changes if material with a high relative permeability, for example, fine particle ferrites or metal particles that are embedded in the press-against part 2, approach the sensor 6 due to deformation of the press-against part 2.

Deformation sensors 6 of this type allow a deformation of the press-against part 2 to be measured in a non-contacting manner such that the risk of adverse effects due to contamination can be prevented. The deformation sensor 6 can be arranged protected in a component of the puncturing device 1, for example, in a support ring 11 limiting the deformation of the press-against part 2, or it can be coated with a protective layer such that simple cleaning of the puncturing device 1 is feasible without risk of damaging the sensor 6.

Another option for measuring a deformation of the press-against part 2 is to provide the deformation sensor 6 as a resistive sensor. The deformation sensor 6 can, for example, comprise a strain gauge that is carried by the press-against part 2. Since the press-against part 2 becomes strongly deformed, it is important in this case to ensure that the strain gauge is sufficiently flexible such that it does not tear when the press-against part 2 is pressed against a body part.

A strain gauge can also be attached to the bracket 8 of the press-against part 2 or the support ring 11. As a result of the transfer of the press-against pressure, any deformation of the press-against part 2 always effects a deformation of a housing part contacting the press-against part, which can then be measured with a strain gauge. A strain gauge arranged as described is a force sensor. For this reason, one aspect of the invention relates to a puncturing device for generating a puncturing wound for obtaining a sample of body fluid, comprising a rubber-elastic press-against part 2 to be pressed against a body part 3 from which a sample of body fluid is to be taken, and a puncturing element drive 4 for driving a puncturing element 5 that is inserted in the puncturing device 1 to perform a puncturing motion, characterized by a strain gauge as a force sensor for determining whether the press-against part is being pressed against the body part at a pressure that is favorable for obtaining a sample.

In the simplest case, a strain gauge can be incorporated into the press-against part 2 or attached on it in the form of a thin resistor wire or a thin foil made from a resistor material such that a deformation of the press-against part 2 effects a change of the electrical resistance of the strain gauge.

If individual regions of the press-against part 2 carry separate strain gauges, the deformation of individual partial regions of the press-against part can be detected separately. If multiple strain gauges are incorporated into the press-against part 2 or attached on it electrically insulated from each other in multiple layers having different orientation in the form of an extension measuring rosette, it is even feasible, in advantageous fashion, to obtain information about the direction of the deformation. An extension measuring rosette carried by the press-against part 2 can, for example, detect a deformation that proceeds in the direction of pressing by means of a first layer of the extension measuring rosette whose resistor wires or resistor bands extend in the direction of pressing. A deformation transverse to the direction of pressing can be detected by means of another layer of the extension measuring rosette whose resistor wires and/or resistor bands extend in the corresponding transverse direction.

Sensor signals generated by the deformation sensor 6 are analyzed by an electronic analytical unit 7. The analytical unit 7 uses the sensor signals to determine if the deformation of the press-against part 2 is of a favorable extent for obtaining a sample, if, for example, it has reached a minimum deformation and does not exceed a given maximum deformation. Since the deformation sensor 6 detects the elastic deformation of the press-against part 2 by means of an electrical and/or magnetic measurement, the deformation can even be quantified, which is a major advantage over mechanical force measuring devices, in which a limit switch is actuated by a spring.

If multiple deformation sensors 6 are used, as in the exemplary embodiment shown, which each measure the elastic deformation of a partial region of the press-against part 2, the analytical unit 7 can in addition determine if an unfavorable orientation of the body part 3 with respect to the puncturing device 1 pressed against it is present. For example, if the deformation values of the various partial regions of the press-against part 2 as detected by the individual deformation sensors 6 deviate from each other by more than a given extent, for example 30%, it can be concluded therefrom that the body part 3 is being pressed against the press-against part 2 at an unfavorable angle.

A result that is determined by the analytical unit 7, in particular, whether favorable conditions for obtaining a sample are present, can be displayed to a user by means of a display facility 10. This display can, for example, be in the form of a numerical value, bar diagram, signal light or signal sound. In the simplest case, a green signal light is sufficient to display favorable conditions. One or more further signal lights can signal too little or too much deformation, for example. If multiple deformation sensors 6 are present, as is the case in the exemplary embodiment shown, information of the type, “press-against force too low,” “press-against force too high,” “press-against force uneven,” and “press-against force is favorable” can be signaled to a user such that the user can change the conditions under which the body part 3 is pressed against the press-against part 2 according to need.

In order to reduce the risk of operating errors, the analytical unit 7 can be coupled to the puncturing element drive 4 and trigger a puncture automatically when favorable conditions are present. In this case, it may be favorable to trigger the puncture not immediately upon detection of a favorable deformation, but only if a favorable deformation is detected over a given period of time of, for example, 0.5 to 2 sec. In this way it can be prevented that obtaining a sample is adversely affected by motions of the body part 3 pressed against the device.

Since some users experience automatic triggering of a puncture to be unpleasant for psychological reasons, it is also feasible to connect the analytical unit 7 to a securing facility (not shown) that prevents the triggering of a puncture unless favorable conditions for obtaining a sample are present. Corresponding puncturing devices have a user-actuated triggering element, for example a button. A puncture proceeds only if the triggering element is actuated and the securing facility is released by the analytical unit 7. The puncturing element drive 4 can, for example, be connected to a trigger circuit that comprises a first securing switch that can be actuated by the analytical unit 7 and a second securing switch that can be actuated by the trigger element. If both switches of the trigger circuit are closed, the puncturing element 5 is put into a puncturing motion by the puncturing element drive 4.

The puncturing device 1 shown schematically in FIG. 1 is an integrated system for obtaining and analyzing a sample of body fluid. For this reason, the puncturing element 5 includes a capillary channel that opens into an analytical zone 52 which is treated with test chemicals in the exemplary embodiment shown and therefore undergoes an analyte-concentration-dependent change of color. For analysis, the analytical zone 52 is illuminated by a light source L and reflected radiation is detected by a detector D. The light source L is controlled by a control unit 54 and the signal of the detector D is analyzed by an analytical unit 55. It is preferable for the analytical unit 55 to also control the control unit 54. The analytical unit 55 performs an analysis of the detector signal in order to determine the concentration of the analyte that is present in the body fluid. The analytical result is output by means of an output unit 56, for example by a liquid crystal display.

The analytical unit 7 for analysis of signals of the deformation sensor 6 is shown in FIG. 1 as a unit that is separate from the control unit 54 and the analytical unit 55. However, these units can also be combined, for example, in a single microprocessor. Analogously, the display facility 10 can also be combined with the output unit 56 to form a single display facility.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE NUMBERS

1 Puncturing device
Press-against part
2a Press-against region
2b Lower region of the press-against part 2
2c Clamping rim
2d Edge rim
3 Body part
4 Puncturing element drive
5 Puncturing element
6 Deformation sensor
7 Analytical unit
8 Bracket
10 Display facility
11 Support ring
52 Analytical zone
54 Control unit
55 Analytical unit
56 Output unit
L Light source
D Detector

What is claimed is:

1. A puncturing device for generating a puncturing wound for obtaining a sample of body fluid, comprising:
a rubber-elastic press-against part configured to press against a body part from which a sample of body fluid is to be taken;
a puncturing element disposed in the puncturing device that is movable in a puncturing motion;
a puncturing element drive operable to drive the puncturing element in the puncturing motion; and
an electrical deformation sensor for detecting an elastic deformation of the press-against part by electrical or magnetic measurement.

2. The puncturing device of claim 1, wherein the deformation sensor generates a sensor signal that depends continuously on the deformation of the press-against part.

3. The puncturing device of claim 1, wherein the deformation sensor comprises a resistive sensor.

4. The puncturing device of claim 3, wherein the deformation sensor comprises a strain gauge.

5. The puncturing device of claim 1, wherein the deformation sensor is operable to detect a magnetic field change caused by a deformation of the press-against part.

6. The puncturing device of claim 1, wherein the deformation sensor comprises an inductive sensor.

7. The puncturing device of claim 1, wherein the press-against part contains a magnetic additive.

8. The puncturing device of claim 7, wherein the magnetic additive is ferromagnetic or ferrimagnetic.

9. The puncturing device of claim 1, wherein the electrical deformation sensor comprises multiple deformation sensors, each of which measures the elastic deformation of a partial region of the press-against part, thereby determining whether an orientation of the puncturing device relative to the body part pressed against it is favorable for obtaining a sample.

10. The puncturing device of claim 1, wherein the press-against part is configured such that tissue of a body part pressed against it bulges into the press-against part and becomes surrounded by the elastically-deforming press-against part, wherein an increased body fluid pressure is generated in the surrounded tissue.

11. The puncturing device of claim 1, wherein the deformation sensor detects the deformation of the press-against part without direct contact with the body part pressed against the press-against part.

12. A method of using a puncturing device of the type having a rubber-elastic press-against part and a puncturing element disposed in the puncturing device, comprising:
pressing a body part against the press-against part;
using an electrical deformation sensor to detect the elastic deformation of the press-against part by electrical or magnetic measurement;
evaluating the measurement to determine whether an orientation of the puncturing device relative to the body part pressed against it is favorable for obtaining a sample; and
puncturing the body part if the orientation is determined to be favorable for obtaining a sample.

13. The method of claim 12, further comprising generating a sensor signal that depends continuously on the deformation of the press-against part.

14. The method of claim 12, further comprising using the deformation sensor to detect a magnetic field change caused by a deformation of the press-against part.

15. The method of claim 12, further comprising using multiple deformation sensors, each of which measures the elastic deformation of a partial region of the press-against part, for the step of determining whether an orientation of the puncturing device relative to the body part pressed against it is favorable for obtaining a sample.

16. The method of claim 12, further comprising pressing body tissue against the press-against part such that the body tissue bulges into the press-against part and becomes surrounded by the elastically-deforming press-against part, thereby generating an increased body fluid pressure is generated in the surrounded tissue.

17. A puncturing device for generating a puncturing wound for obtaining a sample of body fluid, comprising:
a deformable press-against part having an opening and configured to press against a body part from which a sample of body fluid is to be taken;
a puncturing element that is movable into the opening in a puncturing motion to puncture a body part;
a puncturing element drive which is operable to drive the puncturing element in the puncturing motion; and
an electrical deformation sensor which is operable to measure the elastic deformation of at least a portion of the press-against part by electrical or magnetic measurement.

18. The puncturing device of claim 17, wherein the electrical deformation sensor is operable to measure whether the elastic deformation exceeds an upper limit.

19. The puncturing device of claim 17, wherein the electrical deformation sensor comprises multiple deformation sensors, each of which measures the elastic deformation of a partial region of the press-against part.

20. The puncturing device of claim 17, wherein the electrical deformation sensor comprises a strain gauge.
